(19) United States
(12) Patent Application Publication
(10) Pub. No.: US 2025/0207043 A1
(43) Pub. Date: Jun. 26, 2025

(54) ISOTHERMAL PROCESS FOR CONVERTING NAPHTHA TO LIGHT PARAFFINS

(71) Applicant: UOP LLC, Des Plaines, IL (US)

(72) Inventors: John J. Senetar, Naperville, IL (US); Bhogeswararao Seemala, Arlington Heights, IL (US)

(21) Appl. No.: 18/975,577

(22) Filed: Dec. 10, 2024

Related U.S. Application Data
(60) Provisional application No. 63/614,230, filed on Dec. 22, 2023.

(51) Int. Cl. C10G 47/30

(52) U.S. Cl. CPC C10G 47/30; C10G 2300/1037; C10G 2400/20

(57) ABSTRACT

A process for converting naphtha is disclosed. The process comprises contacting a naphtha stream and a hydrogen stream with a catalyst in a reactant passage of a reactor to produce a light paraffinic stream. The reactant passage is cooled by indirect heat exchange with a heat transferred media in a cooling passage adjacent to the reactant passage. The light paraffinic stream is separated into an ethane stream and a propane stream.

### FIELD

The field is the conversion of naphtha to light paraffins. The field may particularly relate to converting naphtha to light paraffins.

### BACKGROUND

Light olefin production is vital to the production of sufficient plastics to meet worldwide demand. Paraffin dehydrogenation (PDH) is a process in which light paraffins such as propane and butane can be dehydrogenated to make propylene and butylene, respectively. Dehydrogenation is an endothermic reaction which requires external heat to drive the reaction to completion.

Fluid catalytic cracking (FCC) is another endothermic process that can be tuned to produce substantial propylene. However, not every FCC unit is tuned to make substantial propylene. Also, high propylene FCC units do not make much ethylene; less than 1% of global ethylene supply comes from FCC.

The great bulk of the ethylene consumed in the production of plastics and petrochemicals such as polyethylene is produced by the thermal cracking of hydrocarbons. Steam is usually mixed with the feed stream to the cracking furnace to reduce the hydrocarbon partial pressure and enhance olefin yield and to reduce the formation and deposition of carbonaceous material in the cracking reactors. The process is therefore often referred to as steam cracking or pyrolysis. Ethane oxidative dehydrogenation is a newer catalytic process for converting ethane to ethylene which can be conducted at lower temperatures with lower carbon oxide emissions than steam cracking.

Two types of feeds are typically used for steam cracking. Ethane feed is used in regions where light hydrocarbon gases are prevalent. In regions, where gas is not abundant, naphtha feed is employed for steam cracking. Pyrolytic naphtha cracking has long set the price in the ethylene industry due to higher production cost versus pyrolytic ethane cracking. The world does not currently produce enough ethane to supply the growing demand for ethylene. Therefore, regions lacking ethane supply such as Asia and Europe rely mainly on naphtha cracking to supply ethylene. Naphtha cracking yields only about 30%-35% ethylene with the balance including both relatively high-value by-products comprising propylene, butadiene, and butene-1 and relatively low value by-products comprising pyoil, pygas, and fuel gas. Additional pressures on naphtha cracking including minimum production requirements and environmental concerns have led to the withholding of government approvals in certain regions such as China. The ethylene industry needs a more efficient, economical, and environmentally friendly route to light olefins from naphtha feeds.

### BRIEF SUMMARY

A process for converting naphtha to light paraffins is disclosed. The process comprises contacting a naphtha stream and a hydrogen stream with a catalyst in a reactant passage of a reactor to produce a light paraffinic stream. The reactant passage is cooled by indirect heat exchange with a heat transferred media in a cooling passage adjacent to the reactant passage. The light paraffinic stream is separated into an ethane stream and a propane stream.

### BRIEF DESCRIPTION OF THE DRAWINGS

FIG. 1 is a schematic drawing of a process for converting naphtha in accordance with an exemplary embodiment of the present disclosure.

FIG. 2 is a schematic drawing of a process for converting naphtha in accordance with another exemplary embodiment of the present disclosure.

### DEFINITIONS

The term “communication” means that fluid flow is operatively permitted between enumerated components, which may be characterized as “fluid communication”.

The term “downstream communication” means that at least a portion of fluid flowing to the subject in downstream communication may operatively flow from the object with which it fluidly communicates.

The term “upstream communication” means that at least a portion of the fluid flowing from the subject in upstream communication may operatively flow to the object with which it fluidly communicates.

The term “direct communication” means that fluid flow from the upstream component enters the downstream component without passing through any other intervening vessel.

The term “indirect communication” means that fluid flow from the upstream component enters the downstream component after passing through an intervening vessel.

The term “bypass” means that the object is out of downstream communication with a bypassing subject at least to the extent of bypassing.

As used herein, the term “predominant” or “predominate” or “predominance” means greater than 50%, suitably greater than 75% and preferably greater than 90%.

The term “Cx” is to be understood to refer to molecules having the number of carbon atoms represented by the subscript “x”. Similarly, the term “Cx−” refers to molecules that contain less than or equal to x and preferably x and less carbon atoms. The term “Cx+” refers to molecules with more than or equal to x and preferably x and more carbon atoms.

The term “column” means a distillation column or columns for separating one or more components of different volatilities. Unless otherwise indicated, each column includes a condenser on an overhead of the column to condense and reflux a portion of an overhead stream back to the top of the column and a reboiler at a bottom of the column to vaporize and send a portion of a bottoms stream back to the bottom of the column. Feeds to the columns may be preheated. The top pressure is the pressure of the overhead vapor at the vapor outlet of the column. The bottom temperature is the liquid bottom outlet temperature. Overhead lines and bottoms lines refer to the net lines from the column downstream of any reflux or reboil to the column. Stripper columns may omit a reboiler at a bottom of the column and instead provide heating requirements and separation impetus from a fluidized inert media such as steam. Stripping columns typically feed a top tray and take main product from the bottom.

As used herein, the term “separator” means a vessel which has an inlet and at least an overhead vapor outlet and a bottoms liquid outlet and may also have an aqueous stream outlet from a boot. A flash drum is a type of separator which may be in downstream communication with a separator that may be operated at higher pressure.

The terms “T10” and “T90” are used here to characterize the volatility of a petroleum fraction such as naphtha. T10 and T90 refer to the temperatures for recovery of 10% and 90%, respectively, in distillation of petroleum products at atmospheric pressure using standard method ASTM D86.

### DETAILED DESCRIPTION

In the proposed process, C4-C7 hydrocarbon feed stock is first charged to a “Naphtha to Ethane and Propane” (NEP) unit to convert naphtha in the presence of hydrogen into desirable ethane and propane along. The produced ethane is fed to an ethylene producing unit. These units provide over 75% yield of ethane to ethylene. The produced propane is fed to a propylene producing unit which provides over 85% yield of propane to propylene. The methane by-product from the naphtha conversion unit and the ethane and propane producing units can be used as a fuel including fuel needed to operate ethylene and propylene producing units which operate at elevated temperatures. Unconverted or under-converted C4+ components in the reactor outlet may be separated from the process. In some embodiments, propane may also be recycled to produce additional ethane so as to increase the ratio of ethane to propane from the process.

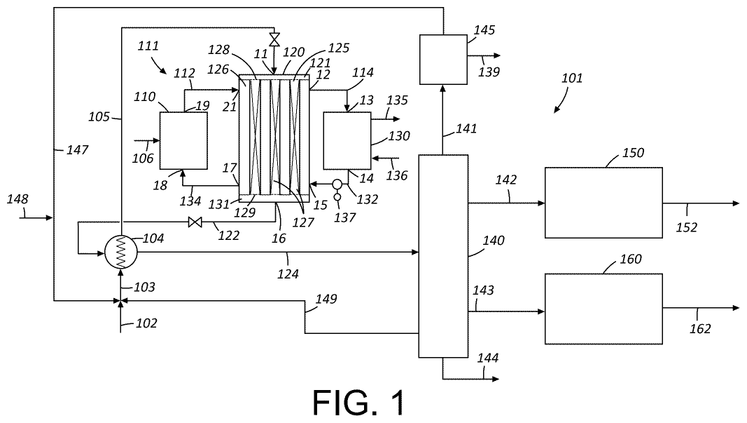

Turning to FIG. 1, an embodiment of the process for converting naphtha 101 is disclosed. In an embodiment, the process 101 comprises a NEP reactor 111, an NEP separation unit 140, an ethane conversion unit 150, and a propane conversion unit 160. A naphtha stream in line 102 may be combined with a hydrogen stream in line 147 and a recycle naphtha stream in line 149 to provide a reactant stream in line 103. The reactant stream in line 103 may be heated with a reactor effluent stream in a heat exchanger 104. A preheated reactant stream is taken from heat exchanger 104 in line 105. In an embodiment, the preheated reactant stream in line 105 is passed to a top of the NEP reactor 111. The preheated reactant stream in line 105 is passed to the NEP reactor 111 through a reactant inlet 11. In an exemplary embodiment, the reactant inlet 11 is located at the top of the NEP reactor 111.

The naphtha stream may comprise C4-C7 hydrocarbons preferably having a T10 between about −10° C. and about 60° C. and a T90 between about 70 and about 180° C. The naphtha feed stream may comprise normal paraffins, iso-paraffins, naphthenes, and aromatics. In an embodiment, the naphtha stream may be heated to a reaction temperature of about 300° C. to about 600° C., suitably between about 325° C. and about 550° C., and preferably between about 350° C. and about 525° C. Overall weight hourly space velocity, defined herein as hourly mass flow rate of feed in stream 102 divided by total catalyst in all reactors should be between about 0.3 to about 20 hr⁻¹, suitably between about 0.5 and about 10 hr⁻¹ and preferably between about 1 to about 4 hr⁻¹. A total pressure should be about 0.1 to about 3 MPa (abs), preferably greater than 1 MPa (abs). Under these conditions, C2-C4 yield from the NEP reactor 111 is consistently in an excess of 80 wt %, while methane yield is less than about 16 wt %, suitably below about 14 wt % and typically below about 12 wt % and preferably no more than 10 wt %.

The hydrogen-to-hydrocarbon molar ratio is important to producing ethane and propane. The hydrogen-to-hydrocarbon ratio should be about 0.3 to about 15 and preferably about 0.5 to about 5. In a further embodiment, the hydrogen-to-hydrocarbon molar ratio may typically be no more than 5, suitably be no more than 3 and preferably be no more than 2. Low hydrogen-to-hydrocarbon ratio promotes desired reaction kinetics which are initiated with dehydrogenation. Hydrogen-to-hydrocarbon ratio may range from about 50% to about 500%, suitably no more than 300% and preferably no more than 200% of stoichiometric requirements to convert naphtha molecules to ethane and/or propane.

The molar ratio of hydrogen to hydrocarbon depends on the feed type including paraffin, naphthene or aromatics, the feed molecular carbon number, and the desired product between predominantly ethane, predominantly propane or ethane and propane of comparable abundance. For example, converting 1 mole of propane to ethane at stoichiometry, the process would require co-feeding 0.5 moles of hydrogen. In practice, the process can operate above or below this stoichiometry of 0.5 such as 0.33 to achieve greater than 40% ethane and less than 15% methane, depending on the process design parameters such as, feed contaminants, reactor type (fixed bed, moving bed, fluidized bed), and regeneration frequency. As the carbon number of feed molecules increases from light naphtha (C4-C7) to full range naphtha (C6-C10) the amount of hydrogen required for the reaction increases. For example, it would require 3.5 moles hydrogen and 2.0 moles of hydrogen to fully convert 1 mole of nonane to ethane and propane, respectively. The disclosed process can operate at three to five times the hydrogen-to-hydrocarbon ratio required to stoichiometrically convert the feed molecules to ethane and propane, respectively. It can also operate at 50% of hydrogen-to-hydrocarbon ratio required to stoichiometrically convert the feed molecules to ethane and propane, respectively. The hydrogen-to-hydrocarbon ratio would also depend on the need to produce petrochemical aromatics such as benzene, toluene, and xylene which are favored at lower hydrogen-to hydrocarbon ratio.

In accordance with the present disclosure, the NEP reactor 111 comprises a reactor vessel 120, a heating unit 110 for heating a heat transfer media, and a heat recovery unit 130 to recover heat from a heated heat transfer media. In an embodiment, the reactor vessel 120 comprises a reactant passage inlet manifold 121, a reactant passage 125, a cooling passage 126 adjacent and perhaps surrounding the reactant passage 125 and a reactant passage outlet manifold 131. The reactant passage 125 comprises NEP catalyst and the reaction takes place inside the reactant passage 125. The reactant passage 125 comprises a reactant passage inlet 128 and a reactant passage outlet 129. The reactant passage inlet manifold 121 may comprise a tube sheet which only admits communication from the reactant passage inlet manifold into the reactant passage inlets 128 of the reactant passages 125. The reactant enters the reactant passage 125 through the reactant passage inlet 128 and after reaction leaves through the reactant passage outlet 129. The reactant passage outlet manifold 131 may comprise a tube sheet which only admits communication into the reactant passage outlet manifold from the reactant passage outlets 129 of the reactant passages 125. In an embodiment, the reactant passage 125 is a tube bundle comprising a plurality of reactor tubes 127. The reactor tubes are filled with the NEP catalysts and the reaction takes place inside the reactor tubes 127. The tube bundle 125 usually has a circular cross section, but the cross section also may be rectangular or of any other shape. In an exemplary embodiment, the plurality of reactor tubes 127 may be vertically disposed within the tube bundle 125.

The NEP catalyst for converting naphtha to ethane and propane may contain a molecular sieve comprising large or medium pore mouths, that is, comprising 10 or 12 member rings, respectively. Examples of suitable molecular sieves include MFI, MEL, MFI/MEL intergrowth, MTW, TUN, UZM-39, IMF, UZM-44, UZM-54, MWW, UZM-37, UZM-8, UZM-8HS. Examples of suitable molecular sieves further include FER, AHT, AEL (SAPO-11), AFO (SAPO-41), MRE, MFS, EUO-1, TON (ZSM-22), MTT (ZSM-23) and UZM-53. Additional molecular sieves with larger pores include FAU, EMT, FAU/EMT intergrowth, UZM-14, MOR, BEA, UZM-50, MTW, ZSM-12. Additional examples include MSE and UZM-35.

MFI is a suitable NEP catalyst. It will be appreciated that ZSM-5 is an MFI-type aluminosilicate zeolite belonging to the pentasil family of zeolites and having a chemical formula of NaₙAlₙSi₉₆-nO₁₉₂·16H₂O (0<n<10). In various embodiments, the ZSM-5 zeolite may comprise a silica-to-alumina molar ratio of 20 to 1000, 20 to 800, 20 to 600, 25 to 400, 25 to 200 or 25 to 80. In various embodiments, the ZSM-5 zeolite may comprise a crystal size in the range of 10 to 600 nm, 20 to 500 nm, 30 to 450, 40 to 400 nm, or 50 to 300 nm.

The NEP catalyst may comprise a bound zeolite. The binder may comprise an oxide of aluminum, silicon, zinc, titanium, zirconium and mixtures of thereof. The binder may comprise a phosphate in the binder or a phosphate of the forenamed oxide binder materials. Preferably, the binder is a silicon oxide. The MFI zeolite may be supported in a silicon oxide containing binder or an alumina containing binder such as aluminum phosphate.

MFI zeolite slurry may be first mixed with a binder in the form of colloidal suspension (sol) and gelling reagent and then dropped into hot oil to make spheres controlled to produce 1/888-inch to about 1/32-inch diameter calcined supports. Alternatively, the zeolite may be mixed with a silicon oxide containing binder and extruded to 1/32 to ¼ inch diameter extrudates. Extrudates may be washed with ammonia to remove sodium ions from the zeolite, dried and calcined to remove the organic structural directing agent (OSDA) from the synthesized zeolite. Optionally, the calcined support may be ammonium-ion exchanged using an ammonium nitrate solution to remove residual sodium ions and dried at about 110° C.

The NEP catalyst comprises a metal on the catalyst. The metal may comprise a transition metal. In a further example, the metal may comprise platinum, palladium, iridium, rhenium, ruthenium and mixtures thereof. The metal may be a noble metal. A modifier metal may also be included on the catalyst. The modifier metal may include tin, germanium, gallium, indium, thallium, zinc, silver and mixtures thereof. The modifier metal should be more concentrated on the binder than on the zeolite. About 0.01 to about 5 wt % of each of the transition metal and the modifier metal may be on the catalyst.

Metal may be incorporated into the binder by evaporative impregnation. A solution of platinum such as tetraamine platinate nitrate or chloroplatinic acid may be contacted with the bound spherical or extrudate supports which have been calcined and ion-exchanged in a rotary evaporator, followed by drying and oxidation.

The NEP catalyst comprises a metal on the bound spherical or extrudate supports of the catalyst. Preferably, more of the metal is on the binder than on the zeolite. At least 60 wt %, suitably at least 70 wt %, preferably at least 80 wt % and most preferably at least 90 wt % of the metal is on the binder. The zeolite and/or the entire NEP catalyst is steamed oxidized to drive the metal off the zeolite. Steaming is preferably effected after the metal is added to the catalyst. The dried, impregnated spherical or extrudate supports may be steam oxidized in air for sufficient time to provide NEP catalysts. Steam oxidation in air at a temperature of about 500° C. to about 650° C. and about 5 mol % to about 30 mol % steam for about 1 to 3 hours may be suitable.

The NEP catalysts must be reduced to activate them for catalyzing the NEP reaction. For example, the catalyst may be reduced in flowing hydrogen at about 500 to about 550° C. for 3 hours before contacting feed.

The catalyst loaded into the reactor may be a single type and formulation or may be a mixture of catalysts with varying functionality and/or activity. Catalyst may be loaded as uniform mixture of two or more catalysts or loaded in separate layers to maximize yield and to further control the rate of heat release across the reactor.

The reactant stream is passed to the reactor vessel 120 through the inlet 11. The reactant passage inlet manifold 124 distributes the reactant stream to the reactant channel inlets 128 and enters the reactant passages 125. When inside the reactant passage, the reactant stream of naphtha stream and hydrogen contacts the NEP catalyst present inside the reactant passages 125 and a light paraffinic stream is produced. The chemistry of NEP process is exothermic, requiring temperature management to prevent runaway reactions. So, a cooling passage 126 is provided surrounding or adjacent the tube bundle 125 to recover the heat of reaction from the tube bundle 125.

In accordance with the present disclosure, a heat transfer media may be passed to the cooling passage 126 to recover the heat of reaction. The heat transfer media indirectly flows in the cooling passage 126 adjacent to the reactant passages 125 and indirectly heat exchange with the reactant present inside the reactant passage 125 to recover the heat of reaction. In an embodiment, the cooling passage may be the shell side of a shell and tube heat exchanger with the reactant passages being the tube side. The cooling passage 126 comprises a heat exchange inlet 21 for receiving the heat transfer media and a heat exchange outlet 12 for withdrawing the heat transfer media after heat exchange. A heat transfer media in line 112 is passed to the cooling passage 126 via the heat exchange inlet 21. In an exemplary embodiment, the heat transfer media in line 112 is a preheated heat transfer media. After heat exchange, the heat transfer media is heated by absorbing the heat of reaction emitted from the reactant passages 125. This heat can be recovered from the heated heat transfer media. A heated heat transfer media may be removed from the cooling passage 126 through the heat exchange outlet 12 in line 114. The heated heat transfer media in line 114 may be passed to the heat recovery unit 130. The heat recovery unit 130 has an inlet 13 for receiving the heated heat transfer media in line 114 and an outlet 14 for withdrawing the heat transfer media from the heat recovery unit 130 after recovering the heat from the heat transfer media. In an aspect of the present disclosure, the heat recovery unit 130 comprises generating steam from the heated heat transfer media in line 114 by indirect heat exchange with water to recover heat from the heat exchange media. In an exemplary embodiment, a boiler feed water stream in line 136 is passed to the heat recovery unit 130. An indirect heat exchange of the boiler feed water stream with the heated heat transfer media in the heat recovery unit 130 produces steam. The produced steam is removed from the heat recovery unit 130 in line 135. A cooled heat transfer media is withdrawn from the outlet 14 of the heat recovery unit 130 in line 132. The cooled heat transfer media in line 132 is at a lower temperature as compared to the temperature of the heat transfer media in line 114. The cooled heat transfer media in line 132 is passed back to the cooling passage 126 perhaps via pump 137 to further recover heat from the reactant and provide cooling. The cooling passage 126 further comprises a subsequent heat exchange inlet 15 and a subsequent heat exchange outlet 17. The cooled heat transfer media in line 132 is passed back to the cooling passage 126 via the subsequent heat exchange inlet 15. The cooled heat transfer media is heated after indirect heat exchange with the reactant in the reactant passages 125. A subsequently heated heat transfer media is removed from the cooling passage 126 via the subsequent heat exchange outlet 17 in line 134.

In accordance with the present disclosure, the heat transfer media may be provided to the cooling passage 126 at a temperature or phase to provide an efficient cooling to the reactant passage. In an aspect of the present disclosure, a heating source or a heating unit 110 is provided to heat the heat transfer media to a predetermined temperature before passing it to the cooling passage 126 via the heat exchange inlet 21. The heated heat transfer media in line 134 is passed to the heating unit 110 via the inlet 18. A fresh heat transfer media taken from a heat transfer media tank in line 106 is also passed to the heating unit 110. The heated heat transfer media in line 134 and the fresh heat transfer media in line 106 are heated in the heating unit 110 to provide a preheated heat transfer media having a predetermined temperature in line 112. The addition of fresh heat transfer media in line 106 may be once through or it can be added periodically to the process 101.

A specific temperature or, if desired, a specific temperature profile along the reactor tubes is required for the majority of exothermic and/or gas phase reactions so as to obtain optimum results in terms of yield and selectivity. The temperature control with the aid of a heat transfer media which influences the temperature of the reactor tubes is particularly significant. A variety of heat transfer media are suitable for resolving that task.

In accordance with the present disclosure, the heat transfer media may comprise one or more salt or mixture thereof. In an exemplary embodiment, the heat transfer media comprises one or more salts selected from sodium nitrite, sodium nitrate, and potassium nitrate. In another exemplary embodiment, the heat transfer media comprises one or more carbonate salts. The salt or the salt mixtures have the advantage of offering better thermal stability. Moreover, they are distinctly less expensive than other media such as ionic liquids-a decisive factor when it comes to large reactors which measure several meters in diameter and length. The composition of the salt may vary and the melting points differ accordingly. The preferred heat transfer media may have a melting point in the range between 100° C. and 450° C. In an embodiment, a mixture of potassium nitrate, sodium nitrate, and sodium nitrite, preferably as a cutectic composition may be used as heat transfer media. This mixture is known as “high temperature salt” (HTS). Its melting point is about 142° C.

For circulation of the heat transfer media in the reactor, the heat transfer media should be kept at a temperature above the melting point for good flow behavior. To achieve that, the heat transfer media preferably is heated outside of the reactor and then introduced into the reactor at elevated temperature. To prevent it from cooling, which would present a risk of the heat transfer media solidifying, first of all the temperature thereof must be well above the melting point. Secondly, the reactor must be preheated at least to the melting temperature.

Thirdly, the connecting lines must be thermally insulated and heated. Fourthly, the filling step must be carried out quickly, and fifthly, the heat transfer media filled into the reactor must be thoroughly circulated and its temperature level maintained or raised still further by heating unit. The heat-up steps performed must not elevate the heat of the reactor too rapidly so as to prevent it from becoming heated unevenly which would result in inadmissible thermal stresses.

The heating unit 110 may be provided for heating the heat transfer media in line 106. The heating unit 110 may be operated in different manners. It may be a heat exchanger which is heated by steam, vapor, or flue gas, or it may be fired. The preferred heating unit 110 is an electrical heating unit 110, an “E-heater.” After its heat-up, the hot heat transfer media returns into the reactor. The means and method steps described below serve to accomplish the above.

In an exemplary embodiment, a molten heat transfer media may be taken in line 112 from the heating unit 110 and passed to the heat exchange inlet 21 of the cooling passage.

In another exemplary embodiment, the heat recovery unit 130 is a cooler.

After paraffin conversion, a light paraffinic stream is discharged from the outlet 129 of the reactant passage. The light paraffinic stream is withdrawn in line 122 from the outlet 16 of the reactor vessel 120. The NEP reactor 111 has a maximum temperature difference of about 10° C. to about 25° C. between the temperature at the inlet 11 and the outlet 16. The light paraffinic stream may comprise at least about 40 wt % ethane or at least about 40 wt % propane or at least about 70 wt % and preferably at least about 80 wt % ethane and propane. The ethane to propane ratio can range from about 0.1 to about 5. The light paraffinic stream can have less than about 16 wt %, suitably less than about 14 wt %, more suitably less than about 12 wt %, preferably less than about 10 wt %, methane.

The light paraffinic stream may be cooled and fed to the NEP separation unit 130.

In an aspect, the light paraffinic stream in line 122 is heat exchanged in the heat exchanger 104 with the reactant stream in line 103 and a cooled light paraffinic stream is formed. The cooled light paraffinic stream is taken in line 124 and passed to NEP separation unit 140.

In accordance with the present disclosure, the process 101 may comprise more than one NEP reactor 111.

The light paraffinic stream may be cooled and fed to an NEP separation unit 130. The NEP separation unit 140 may be a fractionation column or a series of fractionation columns and other separation units that may separate the light paraffinic stream in line 124 into the hydrogen stream in line 147, an ethane stream having a predominance of ethane in line 142, a propane stream having a predominance of propane in line 143 and a heavy aromatics stream in line 144. The NEP reactor 111 of the present disclosure may not need an aromatics recycle stream to maintain the temperature within the reactant passage 125. The NEP separation unit 140 may comprise a demethanizer column that separates the light paraffin stream into a gas stream in an overhead line and a C2+ paraffin stream in a bottoms line. The gas stream may be taken in line 141 and may be sent to a hydrogen purification unit 145 such as a PSA unit to recover hydrogen in line 147 for recycle to the NEP reactor 120. Additional hydrogen make-up gas in line 148 may be added to the recovered hydrogen stream in line 147 and passed to the NEP reactor 120. Remaining methane may be taken in line 139 from the hydrogen purification unit and may be used for fuel gas. The C2+ paraffin stream may then be fed to a deethanizer column to produce the ethane stream in a deethanizer overhead line 142 and a C3+ paraffin stream in a deethanized bottoms line. The C3+ paraffin stream may then be fed to a depropanizer column to produce the propane stream in a depropanizer overhead line 143 and the heavy paraffin stream which may comprise C4+ hydrocarbons. The NEP separation unit 140 may take other forms.

For example, the NEP separation unit 140 may omit a demethanizer column and the light paraffinic stream in line 124 may feed a deethanizer column which produces a C2− stream in a deethanizer overhead line. The C2− stream can be separated in the hydrogen purification unit to recover a hydrogen stream in line 147 while residual ethane and methane from the hydrogen purification unit can comprise or supplement the ethane stream in line 142. The hydrogen purification unit may comprise a membrane unit and the hydrogen recovered from the membrane unit may be further purified in an absorption column before it is recycled to the NEP reactor 111 in line 147. In an additional alternative, the C2− stream from the deethanizer column may be charged to an ethylene producing unit 150 in which ethane is converted to ethylene but methane and hydrogen rides through inertly to be recovered in a downstream ethylene recovery unit. In an aspect, an unconverted naphtha may be separated in the NEP separation unit 140 and recycled back to the NEP reactor 120.

In an embodiment, an unconverted naphtha stream is also recovered from the NEP separation unit 140 in line 149. The unconverted naphtha stream in line 149 is recycled to the NEP reactor 120. In an exemplary embodiment, the unconverted naphtha stream in line 149 comprises C4+ hydrocarbons.

The ethane stream in line 142 may be charged to an ethylene producing unit 150 in which ethane in the ethane stream is converted into ethylene. In an embodiment, the ethylene producing unit 150 is a steam cracking unit. The ethane stream in line 142 may be cracked under steam in a furnace to produce a cracked stream including an ethylene stream 152. The ethane stream may be charged to the ethane steam cracking unit in the gas phase. The ethane steam cracking unit may preferably be operated at a temperature of about 750° C. (1382° F.) to about 950° C. (1742° F.). The cracked stream exiting the furnace of the ethane steam cracking unit may be in a superheated state. One or more quench columns, or other devices known in the art, but preferably an oil quench column and/or a water quench column, may be used for quenching or separating the cracked stream into a plurality of cracked streams. The ethane steam cracking unit may further comprise additional distillation columns, amine wash columns, compressors, expanders, etc. to separate the cracked stream into cracked streams rich in individual light olefins the most predominant of which is the ethylene stream in line 152. The ethylene stream may comprise a yield of at least 75 wt %, preferably at least 80 wt %, ethylene based on the ethane stream in line 142. Among the other components in the cracked stream exiting the ethane steam cracking, ethylene producing unit 150 may be hydrogen, methane, propylene, butene, and pyrolysis gas. Each of these components may be recovered and further processed.

The ethylene stream and a propylene stream from the ethylene producing unit 150 may be recovered or transported to polymerization plants, chemical plants or exported. A butene stream may be recovered and used to produce plastics or other petrochemicals by processes such as polymerization or exported. Product recovery of at least 50 wt %, typically at least 60 wt % and suitably at least 70 wt % of valuable ethylene, propylene, and butylene products is achievable from the ethane steam cracking unit 150 based on the ethane stream in line 142.

The propane stream in line 143 may be charged to a propylene producing unit 160 in which propane in the propane stream is converted into propylene. The propylene producing unit 160 may be a propane dehydrogenation (PDH) unit. PDH catalyst is used in a dehydrogenation reaction process to catalyze the dehydrogenation of propane. The conditions in the dehydrogenation reactor may include a temperature of about 500 to about 800° C., a pressure of about 40 to about 310 kPa (abs) and a catalyst to oil ratio of about 5 to about 100.

The dehydrogenation reaction may be conducted in a fluidized manner such that gas, which may comprise the reactant paraffins with or without a fluidizing inert gas, is distributed to the reactor in a way that lifts the dehydrogenation catalyst in the reactor vessel while catalyzing the dehydrogenation of paraffins. During the catalytic dehydrogenation reaction, coke is deposited on the dehydrogenation catalyst leading to reduction of the activity of the catalyst. The dehydrogenation catalyst must then be regenerated in a regenerator. The regenerator may combust coke from the dehydrogenation catalyst and fuel gas to ensure sufficient enthalpy in the dehydrogenation reactor to promote the endothermic reaction.

The dehydrogenation catalyst selected should minimize cracking reactions and favor dehydrogenation reactions. Suitable catalysts for use herein include an active metal which may be dispersed in a porous inorganic carrier material such as silica, alumina, silica alumina, zirconia, or clay. An exemplary embodiment of a catalyst includes alumina or silica-alumina containing gallium, a noble metal, and an alkali or alkaline earth metal.

The catalyst support comprises a carrier material, a binder and an optional filler material to provide physical strength and integrity. The carrier material may include alumina or silica-alumina. Silica sol or alumina sol may be used as the binder. The alumina or silica-alumina generally contains alumina of gamma, theta and/or delta phases. The catalyst support particles may have a nominal diameter of about 20 to about 150 micrometers with the average diameter of about 70 to about 85 micrometers. Preferably, the surface area of the catalyst support is about 85 to about 140 m²/g.

The fluidized dehydrogenation catalyst may comprise a dehydrogenation metal on a support. The dehydrogenation metal may be a one or a combination of transition metals. A noble metal may be a preferred dehydrogenation metal such as platinum or palladium. Gallium is an effective metal for paraffin dehydrogenation. Metals may be deposited on the catalyst support by impregnation or other suitable methods or included in the carrier material or binder during catalyst preparation.

The acid function of the catalyst should be minimized to prevent cracking and favor dehydrogenation. Alkali metals and alkaline earth metals may also be included in the catalyst to attenuate the acidity of the catalyst. Rare earth metals may be included in the catalyst to control the activity of the catalyst. Concentrations of 0.001% to 10 wt % metals may be incorporated into the dehydrogenation catalyst. In the case of the noble metals, it is preferred to use about 10 parts per million (ppm) by weight to about 600 ppm by weight noble metal. More preferably it is preferred to use about 10 to about 100 ppm by weight noble metal. The preferred noble metal is platinum. Gallium should be present in the range of 0.3 wt % to about 3 wt %, preferably about 0.5 wt % to about 2 wt %. Alkali and alkaline earth metals may be present in the range of about 0.05 wt % to about 1 wt %.

Regenerated catalyst may be contacted with the propane stream in line 143 perhaps with a fluidizing gas to lift the propane stream and dehydrogenation catalyst up a riser while dehydrogenation occurs. Above the riser spent dehydrogenation catalyst and propylene product may be separated by a centripetal separation device. Propylene product gas may be quenched with a cooling fluid to prevent over reaction to undesired by-products. Separation of the propylene product from the PDH effluent stream in line 162 may include quench contacting and fractionation to produce a propylene product stream. Unreacted propane may be recycled to the dehydrogenation reactor and lighter gases may be recycled to the regenerator as fuel gas to be combusted to provide enthalpy for the reaction.

The propylene producing unit may also employ a catalytic moving bed reactor. The reactor section may comprise several radial flow reactors in parallel or series heated by charge and interstage heaters. The propane stream perhaps with added hydrogen flows in each dehydrogenation reactor from a screened center pipe through an annular dehydrogenation catalyst bed to an outer effluent annulus. Flow may be in the reverse fashion. The dehydrogenation catalyst may comprise a noble metal or mixtures thereof, a modifier selected from the group consisting of alkali metals or alkaline-earth metals and mixtures thereof, a component selected from the group consisting of tin, germanium, lead, indium, gallium, thallium, and mixtures thereof, and a porous support forming a catalyst particle. The catalyst support may comprise oil dropped alumina spheres.

Dehydrogenation conditions may include a temperature of from about 400 to about 900° C., a pressure of from about 0.01 to 10 atmospheres absolute, and a liquid hourly space velocity (LHSV) of from about 0.1 to 100 hr−1. The pressure in the dehydrogenation reactor is maintained as low as practicable, consistent with equipment limitations, to maximize chemical equilibrium advantages. Spent dehydrogenation catalyst in the annular catalyst bed may be withdrawn from the bottom of the bed, forwarded to a regenerator to combust coke from the catalyst with air at about 450 to about 600° C. Noble metal on the catalyst may be redispersed by an oxyhalogenation process, dried and returned to the top of the dehydrogenation catalyst bed as regenerated dehydrogenation catalyst.

Dehydrogenation effluent from the propylene producing unit 40 may be cooled, compressed, dried and hydrogen is cryogenically separated from the hydrocarbons with a net gas purity of 85 to 93 mol % hydrogen. Hydrocarbon liquid is selectively hydrogenated to convert diolefins and acetylenes and the hydrocarbon liquid is fractionated in a deethanizer column to remove ethane and propylene is split from propane in a propane-propylene splitter column to provide polymer-grade propylene. Propane may be recycled as feed to the propylene producing unit 40.

The heavy stream which may be taken from a bottom of a depropanizer column in line 144. In an aspect, the heavy stream in line 144 may comprise greater than 98% aromatics.

In accordance with the present disclosure, the disclosure process with the NEP reactor 111 has various benefits/advantages including easy start-up, simple and easier implementation vis a vis heat exchange and heating, produces less methane, leads to higher yield of C2 plus C3, and a simplified operation and control.

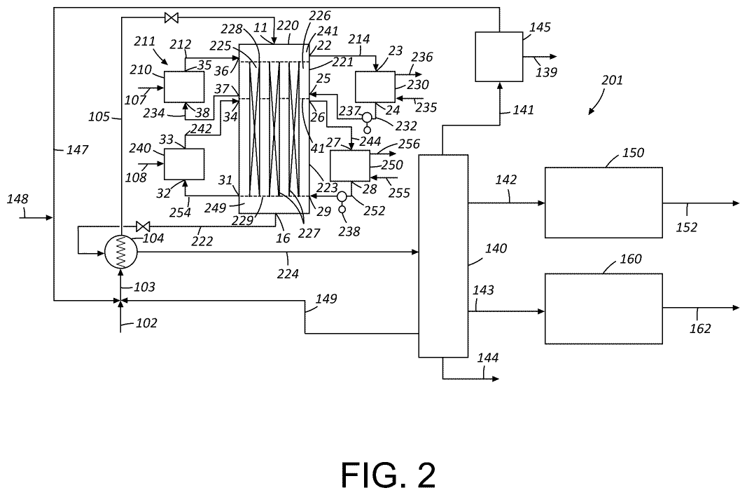

We have found that much of the heat release in the NEP reaction initially in the reaction. Hence, it is important to carefully manage the exotherm early in the NEP reaction. Hence, we provide a further embodiment which utilizes a heat exchange zone just for the inlet region to the NEP catalyst bed. FIG. 2 shows an alternative embodiment to the embodiment of FIG. 1 which employs an NEP reactor 211 having a first zone and a second zone. Elements in FIG. 2 with the same configuration as in FIG. 1 will have the same reference numeral as in FIG. 1. Elements in FIG. 2 which have a different configuration as the corresponding element in FIG. 1 will have the same reference numeral but designated with a prime symbol (′). The configuration and operation of the embodiment of FIG. 2 is essentially the same as in FIG. 1 with the following exceptions.

An alternate embodiment of the process for converting naphtha 201 is shown in FIG. 2. The process 201 comprises an NEP reactor 211 having a reactor vessel 220 having the reactant passage 225, the cooling passage 226, and a reactant passage inlet manifold 241 and a reactant passage outlet manifold 249. In an aspect, the cooling passage 226 comprises a first zone 221 and a second zone 223 for providing cooling to the reactant in the reactant passage 225. The first zone 221 and the second zone 223 are separated by a baffle 41 in the cooling passage 226. In an aspect, the reactant passage 225 is a tube bundle comprising a plurality of reactor tubes 227. The reactant passage 225 comprises a reactant passage inlet 228 and a reactant passage outlet 229 for the reactant stream 105. The reactant passage inlet manifold 241 may comprise a tube sheet which only admits communication from the reactant passage inlet manifold into the reactant passage inlets 228 of the reactant passages 225. The reactant passage outlet manifold 249 may comprise a tube sheet which only admits communication into the reactant passage outlet manifold from the reactant passage outlets 249 of the reactant passages 225. In accordance with the present disclosure, the first zone 221 is provided with a dedicated first heating unit 210 and a dedicated first heat recovery unit 230 for the heat transfer media. Similarly, the second zone 223 is provided with a dedicated second heating unit 240 and a dedicated second heat recovery unit 250 for the heat transfer media. In an aspect, the first zone 221 comprises a first heat exchange inlet 36, a first heat exchange outlet 22, a first subsequent heat exchange inlet 25, and a first subsequent heat exchange outlet 37. In another aspect, the second zone 223 comprises a second heat exchange inlet 34, a second heat exchange outlet 26, a second subsequent heat exchange inlet 29, and a second subsequent heat exchange outlet 31.

In an exemplary embodiment, the first zone 221 is located in a top quarter of the reactor height. In another exemplary embodiment, the first zone 221 is located in top 20% of the reactor height. In yet another exemplary embodiment, the first zone 221 is located in a top 1/7th of the reactor height. In another exemplary embodiment, the first zone 221 is located in a top 1/10th of the reactor height.

The reactant stream is passed to the reactor vessel 220 through the inlet 11. The reactant passage inlet manifold 241 distributes the reactant stream to the reactant channel inlets 228 and enters the reactant passages 225. When inside the reactant passage, the reactant stream of naphtha stream and hydrogen contacts the NEP catalyst present inside the reactant passages 225 and a light paraffinic stream is produced. The NEP reactions are exothermic producing heat. So, the cooling passage 226 is provided surrounding or adjacent the reactant passages 225 to recover the heat of reaction from the reactant passages 225.

A fresh first heat transfer media is passed to the first heating unit 210. A heated first heat transfer media is taken in line 212 from the outlet 35 of the first heating unit 210 and passed to the first zone 221 of the cooling passage via the first heat exchange inlet 36 to supply heat to the reactor at start-up. The heat transfer media indirectly flows in the cooling passage 226 adjacent to the reactant passages 225 and indirectly heat exchange with the reactant present inside the reactant passage 225 to recover the heat of reaction. After heat exchange, the heat transfer media absorbs the heat of reaction emitted from the reactant passages 225. This heat can be recovered from the heated heat transfer media which is removed from the first heat exchange outlet 22 in line 214. Heat is recovered from the heated first heat transfer media in line 214 in the first heat recovery unit 230. The heated first heat transfer media enters the first heat recovery unit 230 via the inlet 23. A boiler feed water stream is passed to the first heat recovery unit 230 in line 235. Steam is taken out from the first heat recovery unit 230 in line 236. A cooled first heat transfer media is withdrawn from the outlet 24 and recycled to the first zone 221 of the cooling passage 226 via the subsequent first heat exchange inlet 25 in line 232 perhaps through the pump 237. A subsequently heated first heat transfer media is removed from the first zone 221 of the cooling passage 226 via the subsequent first heat exchange outlet 37 in line 234. The subsequently heated first heat transfer media in line 234 is passed to the first heating unit 210 via the inlet 38 and heated therein.

In an exemplary embodiment, the first heat transfer media in line 101 comprises one or more salts selected from sodium nitrite, sodium nitrate, and potassium nitrate or a mixture thereof. In an aspect, the first heat transfer media in line 101 is a molten first heat transfer media.

Similarly, a fresh second heat transfer media is passed to the second heating unit 240. A heated second heat transfer media is taken in line 242 from the outlet 33 of the second heating unit 240 and passed to the second zone 223 of the cooling passage via the second heat exchange inlet 34. After indirect heat exchange with the reactants, a heated second heat transfer media is removed from the second heat exchange outlet 26 in line 244. Heat is recovered from the heated second heat transfer media in line 244 in the second heat recovery unit 250. The heated second heat transfer media enters the second heat recovery unit 250 via the inlet 27. A boiler feed water stream is passed to the second heat recovery unit 250 in line 255. Steam is taken out from the second heat recovery unit 250 in line 256. A cooled second heat transfer media is withdrawn from the outlet 28 and recycled to the second zone 223 of the cooling passage 226 via the subsequent second heat exchange inlet 29 in line 252 perhaps through the pump 238. A subsequently heated second heat transfer media is removed from the second zone 223 of the cooling passage 226 via the subsequent second heat exchange outlet 31 in line 254. The subsequently heated second heat transfer media in line 254 is passed to the second heating unit 240 via the inlet 32 and heated therein.

In an exemplary embodiment, the second heat transfer media in line 108 comprises one or more salts selected from sodium nitrite, sodium nitrate, and potassium nitrate or a mixture thereof. In an aspect, the second heat transfer media in line 108 is a molten second heat transfer media.

In another exemplary embodiment, the first heat transfer media in line 101 and the second heat transfer media in line 108 may be the same. In yet another exemplary embodiment, the first heat transfer media in line 101 and the second heat transfer media in line 108 may be different.

After paraffin conversion, a light paraffinic stream is discharged from the outlet 229 of the reactant passage. The light paraffinic stream is withdrawn in line 222 from the outlet 16 of the reactor vessel 220. In an aspect, the light paraffinic stream in line 222 is heat exchanged in the heat exchanger 104 with the reactant stream in line 103 and a cooled light paraffinic stream is formed. The cooled light paraffinic stream is taken in line 224 and passed to NEP separation unit 140. Rest of the process is same as previously described.

In accordance with the present disclosure, the process 201 may comprise more than one NEP reactor 211.

The foregoing disclosure provides a process for converting naphtha to ethane and propane feed with an isothermal reactor.

### EXAMPLE

A naphtha feed was used for the test. The composition of the naphtha feed is as below in Table 1:

TABLE 1

| Constituent | Unit | Value |
|---|---|---|
| Paraffin | wt % | 25.0 |
| Isoparaffin | wt % | 71.1 |
| Naphthenes | wt % | 3.5 |
| Aromatics | wt % | 0.4 |
| Naphtha H/C ratio | mol/mol | 2.4 |

A comparative study was performed to demonstrate and compare the current process with an isothermal reactor 111 with an adiabatic process with four reactor stages in series. Both the processes were conducted at the same inlet pressure of 24.1 bara (335 psig), WHSV of 2 l/h, same H2 content in the product of 10 vol %, at same ethane to propane ratio (E/P) of 2.7. The inlet temperatures for both the processes similar in the range of 430° C. to 435° C. For the adiabatic case, a C6-C8 aromatic recycle stream equivalent to 0.4 weight per 1 weight of naphtha feed was introduced to stage 1 to control the exotherm. For the adiabatic case, naphtha feed was distributed as 14%, 22%, 28%, and 36%, respectively for stages 1 through 4. Hydrogen was distributed as 15.6%, 19.8%, 27.1%, and 37.5%, respectively for stages 1 through 4 to achieve similar hydrogen concentration in the reactor effluent from each stage. Results are shown in Table 2 below:

TABLE 2

| | Unit | | Adiabatic | Isothermal |
|---|---|---|---|---|
| Naphtha Feed Rate | kg/h | | 279203 | 279203 |
| | kMTA | | 2234 | 2234 |
| Recycled Unconverted | kg/h | | 13454 | 4887 |
| Naphtha | | | | |
| Total combined | | | 292657 | 284090 |
| Naphtha Feed | | | | |
| Recycle Aromatics | kg/h | | 111681 | 0 |
| Hydrogen Feed Rate | kg/h | | 12125 | 12585 |
| H2/Naphtha | mol/ | | 1.416 | 1.510 |
| | mol | | | |
| WHSV Naphtha | 1/h | | 2 | 2 |
| H2 at Reactor Outlet | vol % | | 10 | 10 |
| Ethane/Propane (E/P) | w/w | | 2.7 | 2.7 |
| Reactor Pressure | bar | | 24.1 | 24.1 |
| | (abs) | | | |
| Catalyst | kg | Stg 1 | 9519 | |
| | kg | Stg 2 | 15688 | |
| | kg | Stg 3 | 34506 | |
| | kg | Stg 4 | 86287 | |
| | | Total | 145999 | 142045 |
| Naphtha Per Pass | % | | 95.9% | 97.9% |
| Conversion: | | | | |
| H2 Conversion: | % | | 79.5% | 82.8% |
| Selectivity: | | | | |
| C1 | wt % | | 13.3% | 13.2% |
| C2 | wt % | | 58.1% | 59.9% |
| C3 | wt % | | 21.5% | 22.2% |
| C4 | wt % | | | |
| Aromatics | wt % | | 7.1% | 4.7% |
| Total | wt % | | 100.0% | 100.0% |
| Ethane + propane | wt % | | 79.6% | 82.1% |
| (E + P) | | | | |
| Inlet Temperature | | | | |
| Stage 1 | ° C. | | 434.3 | 430.0 |
| Stage 2 | ° C. | | 439.0 | |
| Stage 3 | ° C. | | 440.7 | |
| Stage 4 | ° C. | | 436.1 | |
| Outlet Temperature | | | | |
| Stage 1 | ° C. | | 498.7 | 478.0 |
| Stage 2 | ° C. | | 505.0 | |
| Stage 3 | ° C. | | 505.8 | |
| Stage 4 | ° C. | | 503.2 | |

As shown in the results in Table 2, the process with isothermal reactor provided superior results compared to the adiabatic process. The isothermal case offers the advantages of decreased aromatics yield, increased ethane plus propane (E+P) yield, increased hydrogen conversion, and increased per pass naphtha conversion. In addition, due to the increased per pass naphtha conversion for the isothermal case, the combined naphtha feed including recycle is lower than in the adiabatic case resulting in less catalyst at the same WHSV.

### SPECIFIC EMBODIMENTS

While the following is described in conjunction with specific embodiments, it will be understood that this description is intended to illustrate and not limit the scope of the preceding description and the appended claims.

A first embodiment of the present disclosure is a process for converting naphtha

comprising contacting a naphtha stream and a hydrogen stream with catalyst in a reactant passage of a reactor to produce a light paraffin stream; cooling the reactant passage by indirect heat exchange with a heat transferred media in a cooling passage adjacent to the reactant passage; and separating the light paraffin stream into an ethane stream and a propane stream. An embodiment of the present disclosure is one, any or all of prior embodiments in this paragraph up through the first embodiment in this paragraph further comprising a first zone in the cooling passages comprising a first heat exchange inlet and a first heat exchange outlet and a second zone in the cooling passages comprising a second heat exchange inlet and a second heat exchange outlet. An embodiment of the present disclosure is one, any or all of prior embodiments in this paragraph up through the first embodiment in this paragraph, wherein the first zone is located in a top quarter of the reactor height. An embodiment of the present disclosure is one, any or all of prior embodiments in this paragraph up through the first embodiment in this paragraph, wherein the first zone is at a lower temperature than the second zone. An embodiment of the present disclosure is one, any or all of prior embodiments in this paragraph up through the first embodiment in this paragraph, wherein the reactant passage comprises a tube bundle comprising a plurality of tubes filled with the catalyst. An embodiment of the present disclosure is one, any or all of prior embodiments in this paragraph up through the first embodiment in this paragraph, wherein the reactor comprises a reactor shell enclosing the tube bundle and wherein the cooling passage is in shell side of the reactor. An embodiment of the present disclosure is one, any or all of prior embodiments in this paragraph up through the first embodiment in this paragraph, wherein the reactor comprises an inlet for the reactant and an outlet for the reactor effluent, and wherein maximum temperature in the reactor is within 10° C. of a temperature at the outlet for the reactant. An embodiment of the present disclosure is one, any or all of prior embodiments in this paragraph up through the first embodiment in this paragraph, wherein the heat transfer media is in a molten state. An embodiment of the present disclosure is one, any or all of prior embodiments in this paragraph up through the first embodiment in this paragraph further comprising heating the heat transfer media to provide a molten heat transfer media; passing the molten heat transfer media to the cooling passage through a media inlet to contact and cool the reactant passage; taking a contacted molten heat transfer media from the cooling passage through a media outlet; and cooling the contacted molten heat transfer media in a media cooler to provide a cooled molten heat transfer media. An embodiment of the present disclosure is one, any or all of prior embodiments in this paragraph up through the first embodiment in this paragraph, wherein the heat transfer media comprises one or more salts selected from sodium nitrite, sodium nitrate, and potassium nitrate. An embodiment of the present disclosure is one, any or all of prior embodiments in this paragraph up through the first embodiment in this paragraph, wherein the heat transfer media has a melting temperature in the range of about 100° C. to about 450° C. An embodiment of the present disclosure is one, any or all of prior embodiments in this paragraph up through the first embodiment in this paragraph, wherein the naphtha stream is passed to a top of the reactant passage and the cooling media is passed to a side of the reactant passage.

A second embodiment of the present disclosure is a process for converting naphtha comprising contacting a naphtha stream and a hydrogen stream with catalyst in a reactant passage of a reactor having an inlet and an outlet to produce a light paraffin stream; cooling the reactant passage by indirect heat exchange with a heat transfer media in a cooling passage adjacent to the reactant passage; and separating the light paraffin stream into an ethane stream and a propane stream, wherein maximum temperature in the reactor is within 10° C. of a temperature at the outlet for the reactant. An embodiment of the present disclosure is one, any or all of prior embodiments in this paragraph up through the second embodiment in this paragraph further comprising heating the heat transfer media to provide a molten heat media; and passing the molten heat media to an inlet of the cooling passage. An embodiment of the present disclosure is one, any or all of prior embodiments in this paragraph up through the second embodiment in this paragraph further comprising a cooler in communication with an outlet of the cooling passage to cool a molten heat media. An embodiment of the present disclosure is one, any or all of prior embodiments in this paragraph up through the second embodiment in this paragraph, wherein the heat transfer media comprises one or more salts selected from sodium nitrite, sodium nitrate, and potassium nitrate. An embodiment of the present disclosure is one, any or all of prior embodiments in this paragraph up through the second embodiment in this paragraph, wherein the naphtha stream is passed to a top of the reactant passage and the cooling media is passed to a side of the reactant passage.

A third embodiment of the present disclosure is a process for converting naphtha comprising contacting a naphtha stream and a hydrogen stream with catalyst in a reactant passage comprising a tube bundle to produce a light paraffin stream; cooling the reactant passage by indirect heat exchange with a heat transfer media in a cooling passage adjacent to the reactant passage and enclosing the tube bundle; and separating the light paraffin stream into an ethane stream and a propane stream. An embodiment of the present disclosure is one, any or all of prior embodiments in this paragraph up through the third embodiment in this paragraph, wherein maximum temperature in the reactor is within 10° C. of a temperature at the outlet for the reactant. An embodiment of the present disclosure is one, any or all of prior embodiments in this paragraph up through the third embodiment in this paragraph, wherein the heat transfer media is a molten heat transfer media.

Without further elaboration, it is believed that using the preceding description that one skilled in the art can utilize the present disclosure to its fullest extent and easily ascertain the essential characteristics of this disclosure, without departing from the spirit and scope thereof, to make various changes and modifications of the disclosure and to adapt it to various usages and conditions. The preceding preferred specific embodiments are, therefore, to be construed as merely illustrative, and not limiting the remainder of the disclosure in any way whatsoever, and that it is intended to cover various modifications and equivalent arrangements included within the scope of the appended claims.

In the foregoing, all temperatures are set forth in degrees Celsius and, all parts and percentages are by weight, unless otherwise indicated.

1. A process for converting naphtha comprising:
contacting a naphtha stream and a hydrogen stream with a catalyst in a reactant passage of a reactor to produce a light paraffinic stream;
cooling said reactant passage by indirect heat exchange with a heat transferred media in a cooling passage adjacent to said reactant passage; and
separating the light paraffin stream into an ethane stream and a propane stream.

2. The process of claim 1 further comprising a first zone in the cooling passages comprising a first heat exchange inlet and a first heat exchange outlet and a second zone in the cooling passages comprising a second heat exchange inlet and a second heat exchange outlet.

3. The process of claim 2, wherein the first zone is located in a top quarter of the reactor height.

4. The process of claim 2, wherein the first zone is at a lower temperature than the second zone.

5. The process of claim 1, wherein the reactant passage comprises a tube bundle comprising a plurality of tubes filled with the catalyst.

6. The process of claim 4, wherein the reactor comprises a reactor shell enclosing the tube bundle and wherein the cooling passage is in shell side of the reactor.

7. The process of claim 1, wherein the reactor comprises an inlet for the reactant and an outlet for the reactor effluent, and wherein maximum temperature in the reactor is within 10° C. of a temperature at the outlet for the reactant.

8. The process of claim 1, wherein the heat transfer media is in a molten state.

9. The process of claim 1 further comprising:
heating the heat transfer media to provide a molten heat transfer media;
passing the molten heat transfer media to the cooling passage through a media inlet to contact and cool the reactant passage;
taking a contacted molten heat transfer media from the cooling passage through a media outlet; and
cooling said contacted molten heat transfer media in a media cooler to provide a cooled molten heat transfer media.

10. The process of claim 1, wherein the heat transfer media comprises one or more salts selected from sodium nitrite, sodium nitrate, and potassium nitrate.

11. The process of claim 1, wherein the heat transfer media has a melting temperature in the range of about 100° C. to about 450° C.

12. The process of claim 1, wherein the naphtha stream is passed to a top of the reactant passage and the cooling media is passed to a side of the reactant passage.

13. A process for converting naphtha comprising:
contacting a naphtha stream and a hydrogen stream with catalyst in a reactant passage of a reactor having an inlet and an outlet to produce a light paraffin stream;
cooling said reactant passage by indirect heat exchange with a heat transfer media in a cooling passage adjacent to said reactant passage; and
separating the light paraffin stream into an ethane stream and a propane stream,
wherein maximum temperature in the reactor is within 10° C. of a temperature at the outlet for the reactant.

14. The process of claim 13 further comprising:
heating the heat transfer media to provide a molten heat media; and
passing the molten heat media to an inlet of the cooling passage.

15. The process of claim 14 further comprising a cooler in communication with an outlet of the cooling passage to cool a molten heat media.

16. The process of claim 14, wherein the heat transfer media comprises one or more salts selected from sodium nitrite, sodium nitrate, and potassium nitrate.

17. The process of claim 14, wherein the naphtha stream is passed to a top of the reactant passage and the cooling media is passed to a side of the reactant passage.

18. A process for converting naphtha comprising:
contacting a naphtha stream and a hydrogen stream with catalyst in a reactant passage comprising a tube bundle to produce a light paraffin stream;
cooling said reactant passage by indirect heat exchange with a heat transfer media in a cooling passage adjacent to said reactant passage and enclosing the tube bundle; and
separating the light paraffin stream into an ethane stream and a propane stream.

19. The process of claim 18, wherein maximum temperature in the reactor is within 10° C. of a temperature at the outlet for the reactant.

20. The process of claim 18, wherein the heat transfer media is a molten heat transfer media.
